(19) United States
(12) Patent Application Publication
(10) Pub. No.: US 2020/0171446 A1
(43) Pub. Date: Jun. 4, 2020

(54) Mixing Device for Nicotine and Flavoring

(71) Applicant: Lorenzo De Plano, Los Angeles, CA (US)

(72) Inventor: Lorenzo De Plano, Los Angeles, CA (US)

(21) Appl. No.: 16/207,581

(22) Filed: Dec. 3, 2018

(51) Int. Cl. B01F 3/08; A24F 47/00; A61M 11/04

(52) U.S. Cl. CPC B01F 3/0869; A24F 47/008; A61M 15/06; B01F 3/0803; A61M 11/042

(57) ABSTRACT

A mixing device for infusing/blending liquid nicotine with flavored e-juice. The mixing device includes a tubular body, a plunger screw cap, a puncture mechanism, and a capsule pod. The plunger screw cap includes a disk cap and a pod-receiving tube. The puncture mechanism includes a piercing portion and a structural mount. The piercing portion is mounted within the tubular body by the structural mount, oriented towards a bottom end of the tubular body. The piercing portion breaks open the capsule pod. The plunger screw cap is positioned adjacent to the bottom end. The pod-receiving tube holds the capsule pod and is concentrically connected to the disk cap. The pod-receiving tube is concentrically positioned within the tubular body, adjacent to the bottom end. The plunger screw cap is concentrically and slidably mounted along the tubular body to translate the capsule pod towards and away from the puncture mechanism.

### FIELD OF THE INVENTION

The present invention relates generally to devices for mixing liquids. More specifically, the present invention is a mixing device for infusing/blending liquid nicotine with flavored e-juice, traditionally used in electronic cigarettes.

### BACKGROUND OF THE INVENTION

E-juice is a liquid used in electronic cigarettes. Traditional e-juice, also known as e-liquid, is a combination of propylene glycol, vegetable glycerin, nicotine, and flavoring. The propylene glycol and glycerin produce an aerosol vapor in response to being heated up to a certain temperature within the electronic cigarette. The flavoring provides taste and aroma, while the nicotine acts as a stimulant. E-juice is traditionally sold as a mixture with all the ingredients mixed and cured. The present invention provides a means of mixing the ingredients personally. Specifically, the present invention is a means of infusing nicotine into a premade flavor mixture quickly and efficiently.

The present invention provides a means of accurately diluting nicotine within a specific amount of liquid such that any individual making his or her own e-juice can create store-bought safe dosage of nicotine. Additionally, the present invention provides a means of selling nicotine and flavoring as flavored e-juice, which is illegal to sell together in certain locations, countries, and states.

### BRIEF DESCRIPTION OF THE DRAWINGS

FIG. 1 is a perspective view of the present invention.

FIG. 2 is a perspective view of the present invention in an exploded state.

FIG. 3 is a cross-sectional view of the present invention.

FIG. 4 is a partially internal and perspective view of the present invention in the exploded state.

### DETAIL DESCRIPTIONS OF THE INVENTION

All illustrations of the drawings are for the purpose of describing selected versions of the present invention and are not intended to limit the scope of the present invention.

The present invention is a liquid mixing device. Specifically, the present invention is a device for infusing flavored e-liquid with nicotine for use in electronic cigarettes. Traditional e-liquid, e-juice, or vape juice is composed of propylene glycol (PG), vegetable glycerin (VG), food flavoring, water, and comes with or without nicotine. The present invention is described as a means of mixing flavoring with liquid nicotine, essentially creating custom vape juice. Although, this in no way limits the scope of the present invention as the present invention may be used for mixing other substances and or liquids together in a similar fashion.

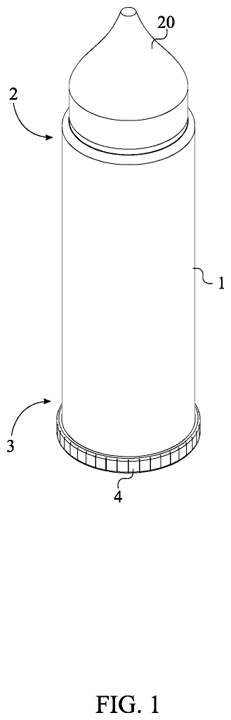

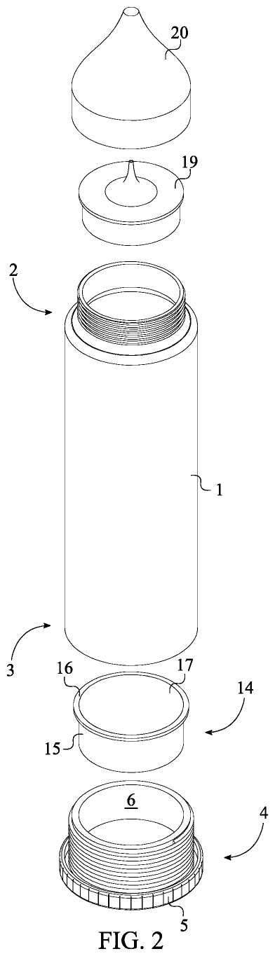

Referring to FIG. 1 and FIG. 2, the present invention comprises a tubular body 1, a plunger screw cap 4, a puncture mechanism 7, a capsule pod 14, a liquid-dispensing nipple 19, and a bottle cap 20. The tubular body 1 forms the body of a container and houses the puncture mechanism 7 and the capsule pod 14. A top end 2 of the tubular body 1 is closed off by the liquid-dispensing nipple 19 and the bottle cap 20. A bottom end 3 of the tubular body 1 is closed off by the plunger screw cap 4. This yields an enclosed vessel for storing and mixing liquids. The capsule pod 14 is filled with a predetermined amount of nicotine, preferably liquid nicotine for more effective mixing. The nicotine concentration or nicotine amount within the capsule pod 14 allows the user to achieve a preferred nicotine level, i.e. mass per volume concentration, within the resulting e-liquid.

The puncture mechanism 7 breaks the capsule pod 14 to release stored contents of the capsule pod 14 within the tubular body 1. The puncture mechanism 7 comprises a piercing portion 8 and a structural mount 10. The piercing portion 8 is a pointed structure designed to cut through, pierce, or otherwise break a relatively thin film. The piercing portion 8 is positioned within the tubular body 1, adjacent to a bottom end 3 of the tubular body 1. Additionally, the piercing portion 8 is oriented towards the bottom end 3. The piercing portion 8 is held in place by the structural mount 10. In particular, the structural mount 10 is mounted in between the tubular body 1 and the piercing portion 8 to support the piercing portion 8. It is preferred that the structural mount 10, the piercing portion 8, and the tubular body 1 are manufactured as a single component.

The plunger screw cap 4 closes off the bottom end 3 of the tubular body 1 and act as the actuation mechanism for pushing the capsule pod 14 against the puncture mechanism 7 to release the contents within the capsule pod 14. For this, the plunger screw cap 4 is positioned adjacent to the bottom end 3 of the tubular body 1 and comprises a disk cap 5 and a pod-receiving tube 6. The disk cap 5 seals the bottom end 3 of the tubular body 1 and acts as a grasping element for the user. It is preferred that a diameter of the disk cap 5 is equal or greater than the tubular body 1. The pod-receiving tube 6 holds the capsule pod 14 within the tubular body 1. Resultantly, the pod-receiving tube 6 is a tube with an external diameter sized to fit within the tubular body 1 and an internal diameter sized receive the capsule pod 14. The plunger screw cap 4 is removably attached to the bottom end 3 of the tubular body 1 such that the user may insert the capsule pod 14 and remove the capsule pod 14 easily. Additionally, the plunger screw cap 4 is concentrically and slidably mounted along the tubular body 1 in order to press the capsule pod 14 against the puncture mechanism 7. Specifically, the pod-receiving tube 6 is concentrically connected to the disk cap 5. The disk cap 5 is positioned concentric and adjacent to the bottom end 3 of the tubular body 1 with the pod-receiving tube 6 being concentrically positioned within the tubular body 1. To attach the capsule pod 14 to the plunger screw cap 4, the user simple places the capsule pod 14 within the pod-receiving tube 6 such that when the plunger screw cap 4 translates along the tubular body 1, the capsule pod 14 translates along a central axis of the tubular body 1; i.e. towards or away from the puncture mechanism 7. In the preferred embodiment of the present invention, the pod-receiving tube 6 is threadably engaged to the tubular body 1 such that rotation of the plunger screw cap 4 translates the plunger screw cap 4 towards or away from the puncture mechanism 7 within the tubular body 1.

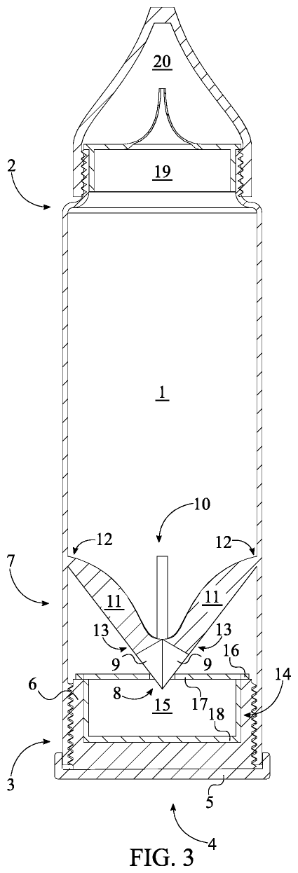

The capsule pod 14 is a container for liquid nicotine with a breakable film for mixing purposes. Specifically, referring to FIG. 3, the capsule pod 14 comprises a capsule tubular body 15, a retaining flange 16, a film disk 17, and a disk end-cap 18. The capsule tubular body 15, the disk end-cap 18, and the film disk 17 delineate a container/vessel for holding liquid nicotine. Specifically, the disk end-cap 18 is terminally and perimetrically connected to the capsule tubular body 15 on one end. Opposite to the disk end-cap 18, the film disk 17 is terminally and perimetrically connected to the capsule tubular body 15. The film disk 17 is a layer of material that is strong enough to contain a liquid within the capsule pod 14 but thin enough to be pierced by the puncturing mechanism. The retaining flange 16 is an annular lip that presses against the pod-receiving tube 6 to properly position the capsule pod 14 within the pod-receiving tube 6. Specifically, the retaining flange 16 is positioned adjacent to the film disk 17. Additionally, the retaining flange 16 is laterally and perimetrically connected to the capsule tubular body 15. To load the capsule pod 14 within the plunger screw cap 4, the capsule tubular body 15 is positioned within the pod-receiving tube 6 with the disk end-cap 18 being positioned adjacent to the disk cap 5. Additionally, the retaining flange 16 is positioned adjacent and pressing against the pod-receiving tube 6, opposite the disk cap 5. To pierce the capsule pod 14, the plunger screw cap 4 is turned about a central axis, thus engaging the threading in between the pod-receiving tube 6 and the tubular body 1 and moving the plunger screw cap 4 and the capsule pod 14 towards the puncture mechanism 7.

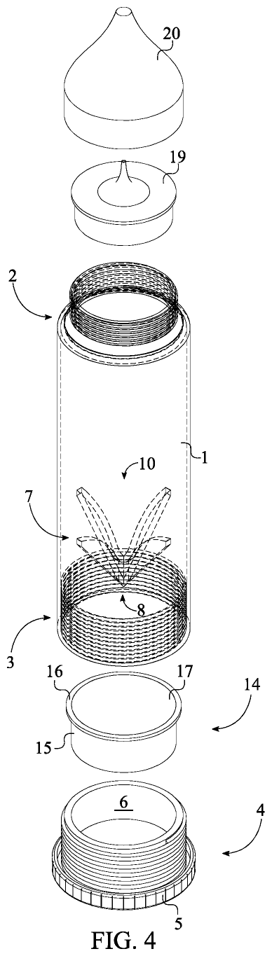

Referring to FIG. 3 and FIG. 4, the structural mount 10 preferably comprises a plurality of structural ribs 11. Each of the plurality of structural beams is a rod, beam, or other similar structural element. The plurality of structural ribs 11 is radially distributed about the tubular body 1 to provide symmetric support to the piercing portion 8. For this, it is preferred that the piercing portion 8 is centrally positioned within the tubular body 1. A first end 12 of each of the plurality of structural ribs 11 is adjacently connected to the tubular body 1. A second end 13 of each of the plurality of structural ribs 11 is adjacently connected to the piercing portion 8. Additionally, in the preferred embodiment of the present invention, the piercing portion 8 comprises a plurality of angled wedges 9. Each of the plurality of angled wedges 9 is a triangle shaped structure that together form a sharp tip. Specifically, each of the plurality of angled wedges 9 is adjacently connected to each other; wherein a specific corner from each of the plurality of angled wedges 9 are connected to each other to form a sharp tip. The plurality of angled wedges 9 is supported by the plurality of structural ribs 11. For this, the plurality of angled wedges 9 is radially distributed about the tubular body 1 with each of the plurality of angled wedges 9 being adjacently connected to a corresponding rib from the plurality of structural ribs 11. It is preferred that the number within the plurality of structural ribs 11 and the number within the plurality of angled wedges 9 is four. Resultantly, space is created in between the plurality of structural ribs 11 that allows contents within the capsule pod 14 to flow through and adequately mix with the contents within the tubular body 1.

Referring to FIG. 4, the liquid-dispensing nipple 19 and the bottle cap 20 close off the top end 2 of the tubular body 1 and allow the user to dispense contents within the present invention. The liquid-dispensing nipple 19 is a disk with an integrated spout. The liquid-dispensing nipple 19 is positioned adjacent to the top end 2 of the tubular body 1 and is concentrically attached within the tubular body 1. Additionally, the liquid-dispensing nipple 19 is oriented away from the bottom end 3, similar to traditional bottle designs. A variety of designs/devices may be used as the liquid-dispensing nipple 19 including, but not limited to, a glass dropper and a screw nipple. The bottle cap 20 is an elongated tubular structure with a closed-off end designed to seal off the top end 2 of the tubular body 1 and the liquid-dispensing nipple 19. The bottle cap 20 is positioned adjacent to the top end 2 of the tubular body 1 with the tubular body 1 being positioned within the bottle cap 20. For easy attachment and detachment, the bottle cap 20 is threadably attached to the tubular body 1.

Although the invention has been explained in relation to its preferred embodiment, it is to be understood that many other possible modifications and variations can be made without departing from the spirit and scope of the invention as hereinafter claimed.

What is claimed is:

1. A mixing device for nicotine and flavoring comprising:
a tubular body;
a plunger screw cap;
a puncture mechanism;
the plunger screw cap comprises a disk cap and a pod-receiving tube;
the puncture mechanism comprises a piercing portion and a structural mount;
the puncture mechanism being positioned within the tubular body, adjacent to a bottom end of the tubular body;
the piercing portion being oriented towards the bottom end of the tubular body;
the structural mount being mounted in between the tubular body and the piercing portion;
the plunger screw cap being positioned adjacent to the bottom end of the tubular body;
the pod-receiving tube being concentrically connected to the disk cap;
the pod-receiving tube being concentrically positioned within the tubular body;
the disk cap being positioned concentric and adjacent to the bottom end of the tubular body; and
the plunger screw cap being concentrically and slidably mounted along the tubular body.

2. The mixing device for nicotine and flavoring as claimed in claim 1 comprising:
the structural mount comprises a plurality of structural ribs;
the plurality of structural ribs being radially distributed about the tubular body;
the piercing portion being centrally positioned within the tubular body;
a first end of each of the plurality of structural ribs being adjacently connected to the tubular body; and
a second end of each of the plurality of structural ribs being adjacently connected to the piercing portion.

3. The mixing device for nicotine and flavoring as claimed in claim 2 comprising:
the piercing portion comprises a plurality of angled wedges;
the plurality of angled wedges being radially distributed about the tubular body;
each of the plurality of angled wedges being adjacently connected to a corresponding rib from the plurality of structural ribs; and
each of the plurality of angled wedges being adjacently connected to each other.

4. The mixing device for nicotine and flavoring as claimed in claim 1 comprising:
a capsule pod;
the capsule pod comprises a capsule tubular body, a retaining flange, and a film disk, and a disk end-cap;
the disk end-cap being terminally and perimetrically connected to the capsule tubular body;
the film disk being terminally and perimetrically connected to the capsule tubular body, opposite to the disk end-cap;
the retaining flange being positioned adjacent to the film disk;
the retaining flange being laterally and perimetrically connected to the capsule tubular body;
the capsule tubular body being concentrically positioned within the pod-receiving tube;
the disk end-cap being positioned adjacent to the disk cap;
the retaining flange being positioned adjacent to the pod-receiving tube, opposite to the disk cap; and
the piercing portion traversing through the film disk.

5. The mixing device for nicotine and flavoring as claimed in claim 1, wherein the pod-receiving tube is threadably attached within the tubular body.

6. The mixing device for nicotine and flavoring as claimed in claim 1 comprising:
a liquid-dispensing nipple;
the liquid-dispensing nipple being positioned adjacent to a top end of the tubular body;
the liquid-dispensing nipple being concentrically attached within the tubular body; and
the liquid-dispensing nipple being oriented away from the bottom end.

7. The mixing device for nicotine and flavoring as claimed in claim 1 comprising:
a bottle cap;
the bottle cap being positioned adjacent to a top end of the tubular body;
the tubular body being positioned within the bottle cap; and
the bottle cap being threadably attached to the tubular body.

8. A mixing device for nicotine and flavoring comprising:
a tubular body;
a plunger screw cap;
a puncture mechanism;
the plunger screw cap comprises a disk cap and a pod-receiving tube;
the puncture mechanism comprises a piercing portion and a structural mount;
the structural mount comprises a plurality of structural ribs;
the puncture mechanism being positioned within the tubular body, adjacent to a bottom end of the tubular body;
the piercing portion being oriented towards the bottom end of the tubular body;
the structural mount being mounted in between the tubular body and the piercing portion;
the plunger screw cap being positioned adjacent to the bottom end of the tubular body;
the pod-receiving tube being concentrically connected to the disk cap;
the pod-receiving tube being concentrically positioned within the tubular body;
the disk cap being positioned concentric and adjacent to the bottom end of the tubular body;
the plunger screw cap being concentrically and slidably mounted along the tubular body;
the plurality of structural ribs being radially distributed about the tubular body;
the piercing portion being centrally positioned within the tubular body;
a first end of each of the plurality of structural ribs being adjacently connected to the tubular body; and
a second end of each of the plurality of structural ribs being adjacently connected to the piercing portion.

9. The mixing device for nicotine and flavoring as claimed in claim 8 comprising:
the piercing portion comprises a plurality of angled wedges;
the plurality of angled wedges being radially distributed about the tubular body;
each of the plurality of angled wedges being adjacently connected to a corresponding rib from the plurality of structural ribs; and
each of the plurality of angled wedges being adjacently connected to each other.

10. The mixing device for nicotine and flavoring as claimed in claim 8 comprising:
a capsule pod;
the capsule pod comprises a capsule tubular body, a retaining flange, and a film disk, and a disk end-cap;
the disk end-cap being terminally and perimetrically connected to the capsule tubular body;
the film disk being terminally and perimetrically connected to the capsule tubular body, opposite to the disk end-cap;
the retaining flange being positioned adjacent to the film disk;
the retaining flange being laterally and perimetrically connected to the capsule tubular body;
the capsule tubular body being concentrically positioned within the pod-receiving tube;
the disk end-cap being positioned adjacent to the disk cap;
the retaining flange being positioned adjacent to the pod-receiving tube, opposite to the disk cap; and
the piercing portion traversing through the film disk.

11. The mixing device for nicotine and flavoring as claimed in claim 8, wherein the pod-receiving tube is threadably attached within the tubular body.

12. The mixing device for nicotine and flavoring as claimed in claim 8 comprising:
a liquid-dispensing nipple;
the liquid-dispensing nipple being positioned adjacent to a top end of the tubular body;
the liquid-dispensing nipple being concentrically attached within the tubular body; and
the liquid-dispensing nipple being oriented away from the bottom end.

13. The mixing device for nicotine and flavoring as claimed in claim 8 comprising:
a bottle cap;
the bottle cap being positioned adjacent to a top end of the tubular body;
the tubular body being positioned within the bottle cap; and
the bottle cap being threadably attached to the tubular body.
